(19) United States
(12) Patent Application Publication
(10) Pub. No.: US 2004/0265445 A1
(43) Pub. Date: Dec. 30, 2004

(54) Microbiocidal control in the processing of poultry

(76) Inventor: Eric W. Liimatta, Baton Rouge, LA (US)

Correspondence Address: EDGAR SPIELMAN, ALBEMARLE CORPORATION, 451 FLORIDA BLVD., BATON ROUGE, LA, 70801, US

(21) Appl. No.: 10/603,130

(22) Filed: Jun. 24, 2003

(51) Int. Cl. A23K 1/00

(52) U.S. Cl. 426/332

(57) ABSTRACT

An eviscerated poultry carcass is subjected to inside-outside washing (IOBW) with a microbiocidal composition which is (a) water having a microbiocidally-effective bromine residual derived from an N,N′-bromochloro-5,5-dialkylhydantoin; or (b) water having a microbiocidally-effective bromine residual derived from alkali or alkaline earth metal bromide and alkali metal or alkaline earth metal hypohalite; or (c) water having a microbiocidally-effective bromine residual derived from a halogen stabilizer, a bromine source, and an alkali metal base or alkaline earth metal base; or (d) a mixture of at least two of (a), (b), and (c). Preferably before the IOBW, the unopened defeathered carcass is washed with such microbiocidal composition. In a preferred multi-staged operation, the microbiocidal composition is applied to the unopened defeathered carcass, to the eviscerated carcass during IOBW, to the eviscerated carcass in the chill tank, and optionally but preferably, to the carcass after removal from the chill tank and before packaging for storage or shipment.

### REFERENCE TO COPENDING APPLICATIONS

Reference is hereby made to U.S. application Ser. No. 10/028,631, filed Dec. 21, 2001, and to commonly-owned U.S. application Ser. No. 10/029,329, filed Dec. 21, 2001, both of which are continuations-in-part of application Ser. No. 09/893,581, filed Jun. 28, 2001, now abandoned. application Ser. No. 10/028,631 is presently owned by another party. Reference is also hereby made to PCT International Application No. PCT/US02/41479, filed Dec. 26, 2002 (presently owned by that other party) in which the United States is one of the designated countries or regions, and to U.S. application Ser. No. ______ [Case SU-7273______], filed _______ [contemporaneously herewith]______ (presently jointly owned with that other party). All four of these applications relate, inter alia, to use of 1,3-dibromo-5,5-dialkylhydantoins as treating agents for water used in the field of animal processing.

### REFERENCE TO OTHER COMMONLY-OWNED APPLICATIONS AND PATENTS

Reference is hereby made to the following commonly-owned applications and patents: application Ser. No. 09/088,300, filed Jun. 1, 1998, now U.S. Pat. No. 6,068,861 issued May 30, 2000; application Ser. No. 09/296,499, filed Apr. 22,1999, now U.S. Pat. No. 6,110,387 issued Aug. 29, 2000; application Ser. No. 09/323,348, filed Jun. 1, 1999, now U.S. Pat. No. 6,303,038 B1 issued Oct. 16, 2001; application Ser. No. 09/404,184, filed Sep. 24, 1999, now U.S. Pat. No. 6,322,822 issued Nov. 27, 2001; application Ser. No. 09/442,025, filed Nov. 17, 1999, now U.S. Pat. No. 6,306,441 issued Oct. 23, 2001; application Ser. No. 09/451,319, filed Nov. 30, 1999; application Ser. No. 09/451,344, filed Nov. 30, 1999, now U.S. Pat. No. 6,352,725 B1 issued March 5, 2002; application Ser. No. 09/456,781, filed Dec. 8, 1999, now U.S. Pat. No. 6,495,169 B1 issued Dec. 17, 2002; application Ser. No. 09/483,896, filed Jan. 18, 2000, now U.S. Pat. No. 6,448,410 B1 issued Sep. 10, 2002; application Ser. No. 09/484,687, filed Jan. 18, 2000, now U.S. Pat. No. 6,508,954 B1 issued Jan. 21, 2003; application Ser. No. 09/484,844, filed Jan. 18, 2000; application Ser. No. 09/484,891, filed Jan. 18, 2000, now U.S. Pat. No. 6,495,698 B1 issued Dec. 17, 2002; application Ser. No. 09/484,938, filed Jan. 18, 2000; application Ser. No. 09/487,816, filed Jan. 18, 2000; application Ser. No. 09/506,911, filed Feb. 18, 2000, now U.S. Pat. No. 6,511,682 B1 issued Jan. 28, 2003; application Ser. No. 09/658,839, filed Sep. 8, 2000, now U.S. Pat. No. 6,375,991 B1 issued Apr. 23, 2002; application Ser. No. 09/663,788, filed Sep. 18, 2000, now U.S. Pat. No. 6,348,219 B1 issued Feb. 19, 2002; application Ser. No. 09/663,948, filed Sep. 18, 2000, now U.S. Pat. No. 6,299,909 B1 issued Oct. 9, 2001; application Ser. No. 09/732,601, filed Dec. 7, 2000, now U.S. Pat. No. 6,506,418 B1 issued Jan. 14, 2003; application Ser. No. 09/775,516, filed Feb. 2, 2001; application Ser. No. 09/778,228, filed Feb. 6, 2001; application Ser. No. 09/785,890, filed Feb. 16, 2001; application Ser. No. 09/893,581, filed Jun. 28, 2001; and application No. 09/974,622, filed Oct. 9, 2001; U.S. application Ser. No. 09/974,626, filed Oct. 9, 2001; U.S. application Ser. No. 10/120,334, filed Apr. 10, 2002; U.S. application Ser. No. 10/269,901, filed Oct. 10, 2002; U.S. application Ser. No. 10/282,290, filed Oct. 28, 2002; U.S. application Ser. No. 10/282,291, filed Oct. 28, 2002; U.S. application Ser. No. 10/313,243, filed Dec. 6, 2002; and U.S. application Ser. No. 10/370,333, filed Feb. 14, 2003.

### BACKGROUND

Contamination of poultry meat products with various pathogens such as species of Listeria, Escherichia, Salmonella, Campylobacter, and others, is a problem that has existed for many years. While various other microbiocidal materials have been investigated for efficacy, the principal antimicrobial substances used in actual practice in poultry processing operations have been sodium hypochlorite and calcium hypochlorite, largely because of their low cost and ready availability.

A need exists for a way of providing more effective microbiocidal control in the processing of poultry than is possible with use of sodium hypochlorite or calcium hypochlorite.

This invention is deemed to fulfill this need.

### BRIEF SUMMARY OF THE INVENTION

Pursuant to one embodiment of this invention, an opened eviscerated carcass is subjected to inside-outside washing with an aqueous microbiocidal composition used pursuant to this invention. This washing can be effected by immersion in an aqueous solution of the microbiocide or by use of exterior spraying of a solution of the microbiocide wherein at least a portion of the spray is directed so that it enters the interior cavity of the carcass. Preferably however, the carcass is subjected to inside-outside washing by use of an inside-outside bird washing (IOBW) apparatus wherein, in addition to exterior washing with an aqueous solution of the microbiocide typically applied by a spray delivery system such as a series or array of nozzles, a spray delivery system such as a probe or bayonet enters the interior cavity and applies therein a pressurized spray of the aqueous solution of the microbiocide to the interior cavity of the carcass.

Pursuant to another embodiment of this invention, an aqueous microbiocidal composition used pursuant to this invention is brought into contact with the defeathered poultry carcass before the carcass has been opened. After a period of time during which carcass remains wet with an aqueous solution of the microbiocide applied to the exterior thereof, the carcass is opened and eviscerated and the opened, eviscerated carcass is subjected to inside-outside washing with an aqueous microbiocidal composition used pursuant to this invention, again preferably by use of an inside-outside bird washing (IOBW) apparatus.

Pursuant to a further embodiment of this invention, an opened, eviscerated poultry carcass is subjected to inside-outside washing with water treated with a microbiocidal composition used pursuant to this invention, again preferably by use of an inside-outside bird washing (IOBW) apparatus, and thereafter the carcass is placed in a chill tank and brought into contact with chill water treated with a microbiocidal composition used pursuant to this invention for a period of time that is at least sufficient for the carcass to reach a pre-selected low temperature.

Pursuant to a another preferred embodiment of this invention, effective microbiocidal control in the processing of poultry is brought about by use of an aqueous solution of the microbiocide in at least three specific, highly important poultry processing stages or stations, whereby without materially affecting productivity, more effective microbiocidal control is achieved as compared to use of the hypochlorite microbiocides. Indeed, this embodiment of the invention makes it possible to minimize individual plant off-line reprocessing operations.

More particularly, pursuant to this preferred embodiment of this invention water treated with a microbiocidal composition used pursuant to this invention is brought into contact with the defeathered poultry carcass before the carcass has been opened. After a period of time during which carcass remains wet with an aqueous solution of the microbiocide applied to the exterior thereof, the carcass is opened and eviscerated. Then the opened and eviscerated carcass is subjected to inside-outside washing by any appropriate method including use of hand-held spraying devices, but preferably by being introduced into an inside-outside bird washer wherein an aqueous microbiocidal composition used pursuant to this invention is brought into contact with the interior and the exterior of the bird, most preferably by use of inside-outside bird washing apparatus (IOBW). Thereafter the carcass is placed in a chill tank and brought into contact with chill water treated with an aqueous microbiocidal composition used pursuant to this invention for a period of time that is at least sufficient for the carcass to reach a pre-selected low temperature. In an especially preferred embodiment, before packaging such carcass for sale, the carcass is again brought into contact with an aqueous microbiocidal composition used pursuant to this invention.

The aqueous microbiocidal compositions used in the practice of the various embodiments of this invention are:

a) water having a bromine residual derived from one or more N,N′-bromochloro-5,5-dialkylhydantoins; or

b) water having a bromine residual derived from at least one alkali metal bromide or alkaline earth metal bromide and at least one alkali metal hypochlorite or alkaline earth metal hypochlorite; or

c) water having a bromine residual derived from a halogen stabilizer, a bromine source and an alkali metal base or alkaline earth metal base; or

d) a mixture or combination of any two of a), b), and c), or of all three of a), b) and c).

Preferably one or more of a), b), and c)—more preferably only one of a), b), and c)—constitute(s) the sole source(s) of microbiocidal activity in the operation(s) being conducted. However this invention includes use in the above operations of one or more of a), b), and c)—more preferably only one of a), b), and c)—and use therewith of one or more other microbiocidal agents that are compatible therewith.

These and other embodiments and features of this invention will be still further apparent from the ensuing description and appended claims.

### FURTHER DETAILED DESCRIPTION OF THE INVENTION

It will be noted that an aqueous microbiocidal composition is used in one or more specified operations in the practice of this invention. The aqueous microbiocidal composition is an aqueous solution of one or more specified microbiocides. In each of the embodiments of this invention the bird carcass is sanitized by employing one or more aqueous microbiocidal composition used pursuant to this invention. Such compositions are typically formed by diluting a more concentrated aqueous solution of a), b), c), or d) above. In fact, the microbiocidal compositions of b) and c) are typically formed in water. On the other hand it is possible with a) to either:

1) mix with water one or more N,N′-bromochloro-5,5-dialkylhydantoins, to form a concentrated solution or slurry which is thereafter diluted with, or added to, additional water to form a solution containing the appropriate use level of bromine residual, or

2) cause the one or more such N,N′-bromochloro-5,5-dialkylhydantoins while in the solid state to come into contact with the water to be used in sanitizing the carcass so that the water receives and contains an appropriate use level of bromine residual from such halohydantoin(s). For example, feeding apparatus or dispensing devices are available for passing a stream of water into and through a bed of microbiocidal tablets or wafers so that a suitable amount of dissolved microbiocide continuously passes into the water. Thus, such apparatus or devices can be used to cause such solid state N,N′-bromochloro-5,5-dialkylhydantoins to come into contact with the water to be used in sanitizing the carcass.

For convenience an aqueous solution of a), b), c), or d) above containing an appropriate microbiocidally effective residual of bromine is simply referred to hereinafter collectively as “aqueous antimicrobial solution a-d”.

In the processing of poultry for consumption as a meat product, this invention comprises in one of its embodiments causing an eviscerated poultry carcass, preferably a mechanically transported series of poultry carcasses, to be subjected to inside-outside washing with an aqueous antimicrobial solution a-d.

While hand spraying equipment can be used, it is preferred to conduct the inside-outside bird washing (IOBW) with apparatus that is adapted to conduct these operations automatically and thoroughly. One such type of apparatus is referred to in U.S. Pat. No. 4,849,237, issued Jul. 18, 1989 wherein carcasses are transported through a trough in which the carcasses can be fully immersed in a cleansing liquid (which pursuant to this invention is an aqueous antimicrobial solution a-d) and wherein rows of nozzles along the bottom are directed to stream jets of cleansing liquid (which pursuant to this invention is an aqueous antimicrobial solution a-d) into the inner body cavity of the carcasses. However, for this purpose it is most preferred to employ apparatus in which a spray probe penetrates the neck cavity from the body cavity or that creates a positive opening in the neck so that the aqueous antimicrobial solution a-d together with contaminants readily drain from the suspended carcass as it is conveyed through the apparatus. Such preferred apparatus will also apply pressurized sprays of the aqueous antimicrobial solution a-d to the exterior of the suspended carcass by means of a manifold or array of spray nozzles so that the exterior of the carcass is also thoroughly cleansed. The exterior of the carcass can be scrubbed by brushes or other flexible scrubbing surfaces as it leaves the apparatus. See for example the apparatus described in U.S. Pat. No. 5,482,503, issued Jan. 9, 1996. Typical apparatus which can be used for such preferred inside-outside bird washing is available from Johnson Food Equipment, Inc. (a member of the Baader Group), 2955 Fairfax Trafficway, Kansas City, Kans. 66115, Telephone 913-621-3366, Web Site www.baader.johnson.com (e.g., Birdwasher 10505-16 with a current indicated capacity of up to 100 birds per minute or Birdwasher 10505-20 with a current indicated capacity of up to 140 birds per minute); and from Cantrell Machine Co., Inc. P.O. Box 757 1400 S. Bradford Street, Gainsville, Ga. 30503, Telephone 770-536-3611, Web Site www.cantrell.com (e.g., Inside/Outside Bird Washer Model No. FIO-515 with a current indicated capacity as a 14 unit machine of 5600 birds per hour).

In a preferred embodiment of this invention in the processing of poultry for consumption as a meat product, this invention comprises the following improvements:

A) causing (i) at least one defeathered poultry carcass, preferably a mechanically transported series of defeathered unopened poultry carcasses, and (ii) an aqueous antimicrobial solution a-d to come into contact with each other, whereby the exterior of at least one carcass is, or the exterior carcasses of the series of carcasses are, wetted by the aqueous antimicrobial solution a-d for a period of time sufficient to provide microbiocidal activity on the wet exterior(s) of the carcass(es);

B) opening and eviscerating the carcass(es) that has been or have been wetted in A),

C) causing an eviscerated poultry carcass, preferably a mechanically transported series of poultry carcasses, to be subjected to inside-outside washing with an aqueous antimicrobial solution a-d.

Another preferred embodiment in the processing of poultry for consumption as a meat product comprises the following improvements:

A) causing (i) aqueous antimicrobial solution a-d and (ii) at least one unopened defeathered poultry carcass to come into contact with each other via either spraying, immersion, or other means of washing, whereby the carcass exterior is wetted by such aqueous antimicrobial solution a-d for a period of time sufficient to provide microbiocidal activity of the wet exterior of the carcass;

B) opening and eviscerating the carcass that was wetted in A);

C) causing the eviscerated carcass to be subjected to inside-outside washing with aqueous antimicrobial solution a-d;

D) causing the carcass that was washed in C) to be placed in a chill tank and brought into contact with chill water which pursuant to this invention is aqueous antimicrobial solution a-d, said carcass being in said chill water for a period of time that is at least sufficient for the carcass to reach a preselected low temperature;

E) causing the chilled carcass to be removed from the chill water, and optionally but preferably

F) before packaging the chilled carcass, causing (i) the chilled carcass and (ii) aqueous antimicrobial solution a-d to come into contact with each other to effect microbiocidal control.

As above, this preferred embodiment is more preferably applied to a mechanically transported series of poultry carcasses. It can be seen that in at least three stages or stations of this preferred embodiment, viz., A), C), and D), and preferably in F) as well, the carcass is sanitized by contact with aqueous antimicrobial solution a-d. Yet, despite the fact that the carcass is sanitized in three or four stages during the overall process, the taste, appearance, and quality of the finished product should not be adversely affected in any significant manner.

The multiple contacting or washing operations when used pursuant to this invention ensure that pathogens such as species of Listeria, Escherichia, Salmonella, Campylobacter, and others, are effectively controlled. Moreover, in large scale bird processing lines where high throughput is essential, the aqueous antimicrobial solution a-d used in these stages or stations should not slow down the line to give the aqueous antimicrobial solution a-d time to act. Thus it should be possible for the processing lines to be operated at conventional speeds. Further, the waters used in the respective stages or stations can each be treated with suitable microbiocidal quantities of a microbiocide referred to above as a), b), c) and d) and thus only one such agent can be used throughout the plant, thus simplifying the purchasing, storage and inventory aspects of the plant operation. Indeed it is deemed possible to use water containing the same microbiocidal concentration of microbiocide a), b), c), or d) in the water going to each of stages A), C), and D), and also in F) as well.

Reference is now made more particularly to the preferred embodiments wherein three or four stages involve washing with aqueous antimicrobial solution a-d. In stage A), the bird carcasses to be treated in the process have already been defeathered upline by means of conventional processing including use of a scalding tank or trough, after which the carcass is typically picked and in some cases singed. In typical automated processing lines, the time for the defeathered carcass to travel from the defeathering stage to the carcass opening and evisceration stage is often in the range of about 20-240 seconds, and this is sufficient time for the washing treatment of this invention to effectively sanitize the exterior of the carcass. This washing or spraying treatment of the invention can involve use of sprays such as by conveying the carcasses through a spraying station or cabinet where the aqueous antimicrobial solution a-d is applied to thoroughly wet the carcasses. Other ways of conducting this washing treatment include immersion of the defeathered carcasses in a bath of aqueous antimicrobial solution a-d, and this can involve, for example, conveying the suspended unopened carcasses through the bath.

In conducting the washing in stage A) the aqueous antimicrobial solution a-d is typically at a temperature of about 5 to about 30° C. The concentration of the particular microbiocidal agent(s) present in the water of aqueous antimicrobial solution a-d provides a bromine residual in the range of about 3 to about 200 ppm (wt/wt) as total bromine, and preferably in the range of about 50 to about 100 ppm (wt/wt) as total bromine. It is not necessary to further rinse the unopened carcass before reaching the carcass opening and evisceration stage. However, a rinse with clear water before opening the carcass can be used if desired.

Stage or station B) involves opening, dismembering, and eviscerating the carcass that was wetted in stage or station A). The steps of opening, dismembering, and eviscerating the carcass can include the removal of at least the head and feet from the carcass, and can be conducted in various sequences. Apparatus for conducting the operation is available from various sources and is widely used in commercial installations.

The inside-outside washing of stage C) can be effected by use of hand operated sprayers. In the typical more highly automated processing plants the washing in C) is effected by use of inside-outside washing apparatus through which the carcass is conveyed. Both the interior cavity and the exterior of the eviscerated carcass are washed with sprays, streams, and/or floods of water. Such interior and exterior washings can be conducted sequentially or concurrently. Here again, apparatus for conducting this overall operation is available in the marketplace and is widely used in commercial installations.

In conducting the inside-outside washing pursuant to this invention the concentration of the particular microbiocidal agent(s) present in the water of aqueous antimicrobial solution a-d provides a bromine residual in the range of about 3 to about 200 ppm (wt/wt) as total bromine, and preferably in the range of about 50 to about 100 ppm (wt/wt) as total bromine. The aqueous antimicrobial solution a-d is typically used at a temperature of about 5 to about 39° C., but can be used at higher temperatures, e.g., up to about 43° C., if desired. Preferred washing apparatus comprises a spray delivery system such as a probe or bayonet which pursuant to this invention applies a pressurized spray of the treated water to the interior cavity of the carcass and another spray delivery system such as a series of nozzles, which system applies the treated water to the exterior of the carcass. In particularly preferred embodiments of this invention aqueous antimicrobial solution a-d applied by the spray delivery system to the interior cavity of the carcass has a higher bromine residual than the bromine residual in the aqueous antimicrobial solution a-d applied by the spray delivery system to the exterior of the carcass. The aqueous antimicrobial solution a-d used for washing the interior cavity of the carcass and the aqueous antimicrobial solution a-d used for washing the exterior of the carcass can be, and usually will be, of the same chemical composition (i.e., both will be composed of a), or b), or c), or d). However, these respective aqueous antimicrobial solutions a-d can have different chemical compositions (e.g., one is from a) and the other is from b), or etc.).

Before reaching the chiller treatment in stage D), the carcass that has been subjected to inside-outside washing can be subjected to further decontamination in stage C), such as further spray rinsing in which water treated pursuant to this invention with bromine residuals at levels as used in the inside-outside washing, is applied at suitable pressures by fixed or articulating nozzles. Such rinsing can be accompanied by use of rotary brushes or other ways of increasing contact such as use of ultrasonic energy. Thereafter the carcass can be rinsed with clear water, if deemed necessary or desirable.

In stage D) the carcass that has been washed in stage C) is placed in a chill tank and brought into contact in the tank with chill water composed of aqueous antimicrobial solution a-d at a suitably low temperature. The water in the chill tank can be fresh or recirculated water, or a combination of both. The recirculated water should be effectively purged of residual impurities from prior usage. Whatever its source, the chill water is treated with a microbiocidally effective amount of the particular microbiocidal agent(s) to form the aqueous antimicrobial solution a-d. The concentration of the particular microbiocidal agent(s) present in the chill water used in the chill tank should provide an aqueous antimicrobial solution a-d having a bromine residual in the range of about 3 to about 200 ppm (wt/wt) as total bromine and preferably in the range of about 50 to about 100 ppm (wt/wt) as total bromine. If monitored on a free bromine basis, the bromine residual in the chill tank would typically be in the range of about 2 to about 125 ppm (wt/wt) as free bromine and preferably in the range of about 10 to about 50 ppm (wt/wt) as free bromine. The temperature of the chill water should be sufficiently low and the residence time of the carcass in the chill water should be sufficient to result in the carcass reaching a temperature in the range of 0 to 7° C., and preferably in the range of 1 to 5° C. The operation in stage D) can involve immersions in more than one chill tank containing water treated pursuant to this invention, and in such case the bromine residuals can be the same or different in successive chill tanks. Also, the chill tank operations can be supplemented by use of cold sprays of either or both of water treated pursuant to this invention and clear water.

After removal from the chill tank, and after rinsing with cold clear water by immersion or spraying, or both, the carcass can be packaged while chilled for storage or transportation under refrigeration. In a preferred embodiment of this invention, after removal from the chill tank the chilled carcass is again washed in stage F) with aqueous antimicrobial solution a-d. The bromine residual in aqueous antimicrobial solution a-d used in stage F) is typically in the range of about 3 to about 200 ppm (wt/wt) as total bromine, and preferably in the range of about 50 to about 100 ppm (wt/wt) as total bromine. This aqueous antimicrobial solution a-d should be cold enough so that the temperature of the carcass does not reach room temperature. Then the carcass is washed with clear water by immersion or spraying, or both, and packaged while chilled for storage or transportation under refrigeration.

It will be appreciated that although the carcass proceeds from stage A) to stage D) or from stage A) to stage F) in the progressions indicated above, one or more intervening steps can be carried out as long as the intervening step or steps do not adversely affect the benefits resulting from use of the process technology of this invention. For example, portions of the carcass, such as the legs and/or wings, can be removed at any suitable time and placed between the stages referred to herein. Also, it is not necessary to conduct all the stages of the process without interruption, although it is preferred to operate on a continuous basis to the extent economically feasible in any given poultry processing facility. For example, it is possible after removing the chilled carcass from the chill tank, to wash the chilled carcass with cold clear water and store the washed and dried carcass under refrigeration on site. Later, when it is desired to package the carcass for sale or shipment, this can be done without further treatment pursuant to this invention. Preferably however after such storage the carcass is subjected to a treatment as in stage F) followed by another wash with cold clear water, and then the washed and dried product is packaged.

When conducting the embodiment of this invention involving only stages A), B), and C) or either of the preferred embodiments of this invention involving stages A), B), C), D), and E) or all of stages A), B), C), D), E), and F), the levels of the bromine residuals of the respective aqueous antimicrobial solutions used in stage A), and C), and whichever, if any, of stages D) and F) are used can be the same, but need not be the same. Using levels of bromine residuals that are the same in all stages simplifies the operation. However, advantages can be achieved by having one or more of these stages at different levels of bromine residuals. This enables use of a higher level of bromine residual in any stage where greater microbial content or more biocidally resistant microbial content is encountered.

Suitable methods for determining “bromine residual” are known and reported in the literature. See for example, Standard Methods For the Examination of Water and Wastewater, 18th Edition, 1992, from American Public Health Association, 1015 Fifteenth Street, NW, Washington, D.C. 20005 (ISBN 0-87553-207-1), pages 4-36 and 4-37; Hach Water Analysis Handbook, Third Edition, 1997, by Hach Company, Loveland Colo., especially pages 1206 and 1207; and Handbook of industrial Water Conditioning, 7th edition, Betz Laboratories, Inc., Trevose, Pa. 19047 (Library of Congress Catalog Card Number: 76-27257), 1976, pages 24-29.

The term “bromine residual” refers to the amount of bromine species present in the treated water available for disinfection. Residuals can be determined as either “total” or “free” depending upon the analytical test method employed. In the present case, the numerical values for bromine residual have been given herein mostly on a total bromine basis. Such values can be monitored by use of the analytical procedure for “total chlorine” given below. However if desired, the bromine residual could be monitored on a “free bromine” basis by using the analytical procedure for “free chlorine” given below. In either case the numerical values obtained are in terms of chlorine and thus such values are multiplied by 2.25 to obtain the corresponding bromine values. Typically the values on a “total bromine” basis on a given sample will be higher than the values on a “free bromine” basis on the same given sample. The important point to understand is that this invention relates to the bromine residual that is actually present in the treated aqueous medium whether the value is determined by use of the total chlorine test procedure or the free chlorine test procedure, but use of the total chlorine test procedure is recommended.

A standard test for determination of low levels of active chlorine which can be used for determination of low levels of active bromine is known as the DPD test and is based on classical test procedures devised by Palin in 1974. See A. T. Palin, “Analytical Control of Water Disinfection With Special Reference to Differential DPD Methods For Chlorine, Chlorine Dioxide, Bromine, Iodine and Ozone”, J. Inst. Water Eng., 1974, 28, 139. While there are various modernized versions of the Palin procedures, the recommended version of the test is fully described in Hach Water Analysis Handbook, 3rd edition, copyright 1997. The procedure for “total chlorine” (i.e., active chlorine) is identified in that publication as Method 8167 appearing on page 379, Briefly, the “total chlorine” test involves introducing. to the dilute water sample containing active halogen, a powder comprising DPD indicator powder, (i. e., N,N′-diethyldiphenylenediamine, KI, and a buffer). The active halogen species present react(s) with KI to yield iodine species which turn the DPD indicator to red/pink. The intensity of the coloration depends upon the concentration of “total chlorine” species (i.e., active chlorine”) present in the sample. This intensity is measured by a calorimeter calibrated to transform the intensity reading into a “total chlorine” value in terms of mg/L Cl₂. If the active halogen present is active bromine, the result in terms of mg/L Cl₂ is multiplied by 2.25 to express the result in terms of mg/L Br₂ of active bromine.

In greater detail, the DPD test procedure is as follows:

1. To determine the amount of species present in the water which respond to the “total chlorine” test, the water sample should be analyzed within a few minutes of being taken, and preferably immediately upon being taken.

2. Hach Method 8167 for testing the amount of species present in the water sample which respond to the “total chlorine” test involves use of the Hach Model DR 2010 calorimeter. The stored program number for chlorine determinations is recalled by keying in “80” on the keyboard, followed by setting the absorbance wavelength to 530 nm by rotating the dial on the side of the instrument. Two identical sample cells are filled to the 25 mL mark with the water under investigation. One of the cells is arbitrarily chosen to be the blank. To the second cell, the contents of a DPD Total Chlorine Powder Pillow are added. This is shaken for 10-20 seconds to mix, as the development of a pink-red color indicates the presence of species in the water which respond positively to the DPD “total chlorine” test reagent. On the keypad, the SHIFT TIMER keys are depressed to commence a three minute reaction time. After three minutes the instrument beeps to signal the reaction is complete. The blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010 and replaced with the test sample to which the DPD “total chlorine” test reagent was added. The light shield is then closed as was done for the blank, and the READ key is depressed. The result, in mg/L Cl₂ is shown on the display within a few seconds. This is the “total chlorine” level of the water sample under investigation. It is to be noted that the test sample may need to be diluted with halogen demand free water in order for the chlorine measurement to be within the measuring range of the instrument. This dilution will need to be taken into account to determine the actual chlorine level of the sample.

3. One method for measuring free chlorine is the Hach Method 8021. This tests for the amount of species present in the water sample which respond to the “free chlorine” test. This test involves the use of the Hach Model DR 2010 calorimeter. The stored program number for chlorine determinations is recalled by keying in “80” on the keyboard, followed by setting the absorbance wavelength to 530 nm by rotating the dial on the side of the instrument. Two identical sample cells are filled to the 25 mL mark with the water under investigation. One of the cells is arbitrarily chosen to be the blank. The blank sample cell is admitted to the sample compartment of the Hach Model DR 2010, and the shield is closed to prevent stray light effects. Then the ZERO key is depressed. After a few seconds, the display registers 0.00 mg/L Cl₂. Then, the blank sample cell used to zero the instrument is removed from the cell compartment of the Hach Model DR 2010. To the second cell, the contents of a DPD Free Chlorine Powder Pillow are added. This is shaken for 10-20 seconds to mix, as the development of a pink-red color indicates the presence of species in the water which respond positively to the DPD “free chlorine” test reagent. Immediately (within one minute of reagent addition) place the prepared sample into the cell holder. The light shield is then closed as was done for the blank, and the READ key is depressed. The result, in mg/L Cl₂ is shown on the display within a few seconds. This is the “free chlorine” level of the water sample under investigation. It is to be noted that the test sample may need to be diluted with halogen demand free water in order for the chlorine measurement to be within the measuring range of the instrument. The dilution will need to be taken into account when determining the chlorine level of the sample.

As made clear at the outset, different specified bromine-based aqueous antimicrobial solutions can be used in the practice of this invention. The aqueous antimicrobial solutions of a) are formed from one or more N,N′-bromochloro-5,5-dialkylhydantoins in which each alkyl group independently contains in the range of 1 to about 4 carbon atoms. Suitable compounds of this type include, for example, such compounds as N,N′-bromochloro-5,5-dimethylhydantoin, N,N′-bromochloro-5-ethyl-5-methylhydantoin, N,N′-bromochloro-5-propyl-5-methylhydantoin, N,N′-bromochloro-5-isopropyl-5-methylhydantoin, N,N′-bromochloro-5-butyl-5-methylhydantoin, N,N′-bromochloro-5-isobutyl-5-methylhydantoin, N,N′-bromochloro-5-sec-butyl-5-methylhydantoin, N,N′-bromochloro-5-tert-butyl-5-methylhydantoin, N,N′-bromochloro-5,5-diethylhydantoin, and mixtures of any two or more of the foregoing. N,N′-bromochloro-5,5-dimethylhydantoin is available commercially under the trade designation Bromicide® biocide (Great Lakes Chemical Corporation). Another suitable bromochlorohydantoin is composed of a mixture of a predominate amount by weight of N,N′-bromochloro-5,5-dimethylhydantoin together with a minor proportion by weight of 1,3-dichloro-5,5-dimethylhydantoin and 1,3-dichloro-5-ethyl-5-methylhydantoin. A mixture of this latter type is available in the marketplace under the trade designation Dantobrom(® biocide (Lonza Corporation) which is believed to contain about 60 wt % of N,N′-bromochloro-5,5-dimethylhydantoin, about 27.4 wt % of 1,3-dichloro-5,5-dimethylhydantoin, about 10.6 wt % of 1,3-dichloro-5-ethyl-5-methylhydantoin, and about 2 wt % of inerts. Most preferred is N,N′-bromochloro-5,5-dimethylhydantoin itself

When a mixture of two or more N,N′-bromochloro-5,5-dialkylhydantoin biocides is used pursuant to this invention, the individual biocides of the mixture can be in any proportions relative to each other. Minor proportions (less than 50 wt %) of mono-N-bromo-5,5-dialkylhydantoin(s) can also be present, either with such mixtures of two or more N,N′-bromochloro-5,5-dialkylhydantoin biocides, or with only one N,N′-bromochloro-5,5-dialkylhydantoin biocide.

Methods for producing such N,N′-bromochloro-5,5-dialkylhydantoins are known and reported in the literature.

The N,N′-bromochloro-5,5-dialkylhydantoin(s) used pursuant to this invention can be blended directly in the water to be used in the various stages referred to herein. For this purpose suitable dispensing devices can be employed that meter into water flowing through the device suitable amounts of the N,N′-bromochloro-5,5-dialkylhydantoin microbiocide(s). Alternatively, predetermined quantities of micronized or finely-divided N,N′-bromochloro-5,5-dialkylhydantoin(s) may be added to water in amounts in excess of the final use level, and the resultant concentrate is thereafter further diluted, preferably with agitation, with one or more different amounts of water to form one or more treated water compositions to be used in the respective stages of the process.

The aqueous antimicrobial solutions of b) are formed from water, at least one alkali metal or alkaline earth metal bromide and at least one alkali metal hypochlorite or alkaline earth metal hypochlorite. The interaction of these components results in an aqueous solution having a suitably high bromine residual, which typically is diluted with additional water to form an aqueous antimicrobial solution a-d. Various suitable alkali metal bromides such as LiBr, NaBr, KBr, etc., and suitable alkaline earth metal bromides, viz., MgBr₂ and CaBr₂ can be used in forming aqueous antimicrobial solutions of b). Preferred for this use is NaBr, especially NaBr from which trace amounts of alcohol such as methanol have been removed. Similarly, various alkali metal hypochlorites or alkaline earth metal hypochlorites can be used. Thus, use can be made of such materials as lithium hypochlorite, sodium hypochlorite, potassium hypochlorite, calcium hypochlorite, magnesium hypochlorite, and the like. Of such hypohalites use of sodium hypochlorite or calcium hypochlorite is most preferred. Several hypochlorite solutions are commercially available as articles of commerce since they are useful as bleaches, as well as intermediates for preparing other useful products. Metal bromides or hypochlorites of Be, Sr, or Ba should not be used because of toxicological concerns. Thus, the term “alkaline earth” as used herein excludes Be, Sr, and Ba.

If an excess amount of the hypochlorite is used relative to the amount of bromide salt used, the resultant solution will contain chlorine-based species as well as the bromine residual. These chlorine-based species are not harmful as long as the requisite quantity of bromine reserve is present in the solution being used. Preferably, any excess of hypochlorite is back-titrated with an aqueous alkali metal hypochlorite or alkaline earth metal hypochlorite so that the halogen reserve in the solution essentially consists of bromine reserve.

The aqueous antimicrobial solutions of c) are formed from water, a halogen stabilizer, a bromine source and an alkali metal base or alkaline earth metal base. These compositions and their preparation are described in numerous patent disclosures, including U.S. Pat. Nos. 6,123,870; 6,156,229; 6,287,473; and 6,423,267. The halogen stabilizers for use in forming these aqueous antimicrobial solutions of c) can be any of the halogen stabilizers described in one or more of the foregoing patents. Examples of such halogen stabilizers are compounds of the group consisting of R—NH₂, R—NH—R¹, R—SO₂—NH₂, R—SO₂—NHR¹, R—CO—NH₂, N—CO—NH—R¹, and R—CO—NH—CO—R¹ where R is a hydroxyl group or an alkyl group or an aromatic group and R¹ is an alkyl group or an aromatic group. The entire disclosure of each such patent is incorporated herein by reference as if fully set forth herein; and thus the materials used, the manner in which they are used, and the conditions under which they are used in forming the aqueous antimicrobial solutions of c) are as set forth in the disclosures of these patents.

Preferred aqueous antimicrobial solutions of c) are those set forth in one or more of U.S. Pat. Nos. 6,068,861; 6,299,909; 6,306,441; 6,322,822; 6,348,219; 6,352,725; 6,375,991; 6,495,169; 6,506,418; and 6,511,682. The entire disclosure of each such patent is incorporated herein by reference as if fully set forth herein, and thus the materials used, the manner in which they are used, the conditions under which they are formed, and the manner in which they are used in forming the aqueous antimicrobial solutions of c) are as set forth in the disclosures of these patents. In these aqueous antimicrobial solutions, active bromine is in a composition containing sulfamate and base as described in this latter group of patents.

Preferred aqueous antimicrobial solutions of c) are biocide compositions comprising water having in solution therein an active bromine content of at least about 100,000 ppm (wt/wt), which active bromine content is a derivative of (i) bromine chloride or a combination of bromine chloride and bromine, and (ii) an aqueous solution of alkali metal salt of sulfamic acid, or (iii) water and an alkali metal salt of sulfamic acid, or (iv) water, an alkali metal base, and sulfamic acid, or (v) any combination of (ii), (iii), and (iv), and in relative proportions of such that the atom ratio of nitrogen to active bromine in said biocide composition is greater than 0.93, and wherein the pH of the biocide composition is at least 10, more desirably at least 12 and still more desirably at least 13.

The aqueous antimicrobial solutions of d) are formed either by mixing together aqueous antimicrobial solutions of any two of a), b), and c), or all three of a), b), and c), or by employing separate aqueous antimicrobial solutions of any two of a), b), and c), or all three of a), b), and c). When employing separate solutions, the separate aqueous antimicrobial solutions can be employed either sequentially in the same operation (e.g., in washing the unopened defeathered carcass, or in the inside-outside washing of the opened carcass, or in the chill tank, or in washing prior to packaging for sale) or individually in these different operations.

Other additives can be used in conjunction with the aqueous antimicrobial solution a-d, provided the other additive or additives are non-toxic, are compatible with the aqueous antimicrobial solution a-d, and do not otherwise detract from the microbiocidal effectiveness of the aqueous antimicrobial solution a-d in any appreciable manner. By “in conjunction with” is meant that in most cases the other additive component(s) are fed separately into the water being used; e.g., the other additives, if susceptible to oxidation by common oxidants, are not mixed directly with a concentrated aqueous microbiocidal solution to be diluted to form an aqueous antimicrobial solution a-d. In general, additives which are compatible with aqueous hypohalite bleach solutions such as certain radical scavengers, chelating agents, pH buffering agents, surfactants, and polymers described in detail in U.S. Pat. No. 6,506,718 may be used, if desired. It is also possible to use one or more wetting agents, hydrotropes, thickeners, defoaming agents, and similar functional additives that meet the above criteria. If used, the amount of each suitable selected additive to be used in conjunction with the microbiocides used pursuant to this invention should be sufficient to provide the property for which it is employed. Recommendations from manufacturers of such other additives are useful as guidelines in this respect.

Various species of poultry can be processed pursuant to this invention. Non-limiting examples of poultry that can be processed include chicken, rooster, turkey, duck, goose, quail, pheasant, ostrich, game hen, emu, squab, guinea fowl, and Cornish hen.

An end result achievable by the practice of this invention is effective reduction or minimization of microbiological contamination of the meat product at all stages of the above-specified operations, and the provision of a meat product in which the taste, sensory quality, appearance, and wholesomeness of the product should not be adversely affected in any material manner by the microbiocidal operations conducted pursuant to this invention. A number of literature references describe suitable methods for testing the qualities of poultry meat products, and any art-recognized procedure can be used to evaluate the taste, sensory quality, appearance, and/or wholesomeness of the product processed pursuant to this invention. One such reference is a paper of A. I. Ikeme, B. Swaminathan, M. A. Cousin, and W. J. Stadelman entitled “Extending the Shelf-Life of Chicken Broiler Meat”, Poultry Science, 1982, 61, 2200-2207.

Although reference is sometimes made above to a “carcass” it is to be understood that in actual operations the process is typically applied to a continuous procession of “carcasses” which are carried on or by conveyor belts which are usually equipped with suitable fastening means. Also, the words “stages” and “stations” are used interchangeably in this description.

Compounds referred to by chemical name or formula anywhere in this document, whether referred to in the singular or plural, are identified as they exist prior to coming into contact with another substance referred to by chemical name or chemical type (e.g., another component, a solvent, or etc.). It matters not what chemical changes, if any, take place in the resulting mixture or solution, as such changes are the natural result of bringing the specified substances together under the conditions called for pursuant to this disclosure. The transformations that take place as the result of bringing these substances together, are usually known to chemists and need no further elaboration.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

All documents referred to herein are incorporated herein by reference in toto as if fully set forth in this document.

This invention is susceptible to considerable variation within the spirit and scope of the appended claims.

That which is claimed is:

1. In the processing of poultry for consumption as a meat product, the improvement which comprises causing an eviscerated poultry carcass to be subjected to inside-outside washing with a microbiocidal composition consisting essentially of
a) water having a microbiocidally-effective bromine residual derived from one or more N,N′-bromochloro-5,5-dialkylhydantoins; or
b) water having a bromine residual derived from at least one alkali or alkaline earth metal bromide and at least one alkali metal hypohalite or alkaline earth metal hypohalite; or
c) water having a bromine residual derived from a halogen stabilizer, a bromine source and an alkali metal base or alkaline earth metal base; or
d) water having a bromine residual derived from a mixture or combination of any two of a), b), and c), or of all three of a), b) and c);
the bromine residual being sufficient to provide microbiocidal activity without significant adverse effect upon the taste, odor, or appearance of the carcass.

2. The improvement as in claim 1 wherein a mechanically transported series of poultry carcasses is automatically transported into apparatus in which the poultry carcass is subjected to said inside-outside washing.

3. The improvement as in claim 2 wherein in said inside-outside washing, the interior cavity of a transported poultry carcass is penetrated by a spray probe so that (i) contaminants together with (ii) microbiocidal composition that is sprayed into the interior cavity of the poultry by the probe, drain from the carcass.

4. The improvement as in claim 3 wherein in said inside-outside washing, pressurized sprays of the microbiocidal composition are applied to the exterior of the carcass so that the exterior of the carcass is thoroughly cleansed, and optionally the exterior of the carcass is also automatically mechanically scrubbed.

5. The improvement as in any of claims 1-4 wherein the microbiocidal composition used is a composition of a).

6. The improvement as in any of claims 1-4 wherein the microbiocidal composition used is a composition of b).

7. The improvement as in any of claims 1-4 wherein the microbiocidal composition used is a composition of c).

8. In the processing of poultry for consumption as a meat product, the improvement which comprises:
A) causing (i) at least one unopened defeathered poultry carcass and (ii) a microbiocidal composition selected from the group consisting of a), b), c), and d) as described in claim 1 to come into contact with each other, via either spraying, immersion, or other form of washing whereby the exterior of said carcass is wetted by such composition for a period of time sufficient to provide microbiocidal activity on the wet exterior of said carcass;
B) opening and eviscerating the carcass that was wetted in A);
C) causing the opened and eviscerated poultry carcass to be subjected to inside-outside washing with a microbiocidally-effective amount of microbiocidal composition selected from the group consisting of a), b), c), and d) as described in claim 1.

9. The improvement as in claim 8 wherein the at least one defeathered poultry carcass in A) is one of a series of unopened defeathered poultry carcasses that are mechanically transported to a station where the poultry carcasses and said microbiocidal composition come into contact with each other; wherein a series of carcasses wetted in A) are mechanically transported to a station where in B) the series of carcasses are opened and eviscerated, and wherein in C) a series of poultry carcasses opened and eviscerated in B) is caused to be subjected to said inside-outside washing.

10. The improvement as in claim 9 wherein in said inside-outside washing, the interior cavity of a transported poultry carcass is penetrated by a spray probe which applies pressurized sprays of said microbiocidal composition to the interior cavity of the carcass so that (i) contaminants together with (ii) microbiocidal composition that is sprayed into the interior cavity of the poultry by the probe, drain from the carcass; and wherein in said inside-outside washing, pressurized sprays of the microbiocidal composition are applied to the exterior of the carcass so that the exterior of the carcass is thoroughly cleansed, and optionally the exterior of the carcass is also automatically mechanically scrubbed.

11. The improvement as in any of claims 8-10 wherein said microbiocidal composition is a composition of a).

12. The improvement as in any of claims 8-10 wherein said microbiocidal composition is a composition of b).

13. The improvement as in any of claims 8-10 wherein said microbiocidal composition is a composition of c).

14. In the processing of poultry for consumption as a meat product, the improvement which comprises:
A) causing (i) a microbiocidal composition selected from the group consisting of a), b), c), and d) as described in claim 1 and (ii) at least one unopened defeathered poultry carcass to come into contact with each other via either spraying, immersion, or other form of washing, whereby the carcass exterior is wetted by such composition for a period of time sufficient to provide microbiocidal activity of the wet exterior of the carcass;
B) opening and eviscerating the carcass that was wetted in A);
C) causing the eviscerated carcass to be subjected to inside-outside washing with a microbiocidally-effective amount of a microbiocidal composition selected from the group consisting of a), b), c), and d) as described in claim 1; and
D) causing the carcass that was washed in C) to be placed in a chill tank and brought into contact with chill water which is composed of a microbiocidally-effective amount of a microbiocidal composition selected from the group consisting of a), b), c), and d) as described in claim 1, said carcass being in said chill water for a period of time that is at least sufficient for the carcass to reach a pre-selected low temperature.

15. The improvement as in claim 14 wherein to cause the contacting in A), said microbiocidal composition is sprayed on said defeathered poultry carcass.

16. The improvement as in claim 14 wherein to cause the contacting in A), said defeathered poultry carcass is immersed in said microbiocidal composition.

17. The improvement as in claim 14 wherein the washing in C) is effected by use of an inside-outside washing apparatus through which the carcass is conveyed.

18. The improvement as in claim 17 wherein said washing apparatus comprises a spray delivery system adapted to apply said microbiocidal composition to the interior cavity of said carcass and another spray delivery system adapted to apply said microbiocidal composition to the exterior of said carcass.

19. The improvement as in claim 14 wherein to cause the contacting in A), said microbiocidal composition is sprayed on said defeathered poultry carcass; and wherein the washing in C) is effected by use of an inside-outside washing apparatus through which the carcass is conveyed.

20. The improvement as in claim 19 wherein said microbiocidal composition is a composition of a).

21. The improvement as in claim 19 wherein said microbiocidal composition is a composition of b).

22. The improvement as in claim 19 wherein said microbiocidal composition is a composition of c).

23. In the slaughter and processing of poultry as a meat product, the improvement which comprises:
A) causing (i) a microbiocidal composition selected from the group consisting of a), b), c), and d) as described in claim 1 and (ii) at least one unopened defeathered poultry carcass to come into contact with each other before the carcass is opened, whereby the carcass exterior is wetted by such microbiocidal composition for a period of time sufficient to provide microbiocidal activity on the wet exterior of the carcass;
B) opening and eviscerating the carcass that was wetted in A);
C) causing the eviscerated carcass to be subjected to inside-outside washing with a microbiocidally-effective amount of a microbiocidal composition selected from the group consisting of a), b), c), and d) as described in claim 1;
D) causing the carcass that was washed in C) to be placed in a chill tank and brought into contact with chill water which is composed of a microbiocidal composition selected from the group consisting of a), b), c), and d) as described in claim 1, said carcass being in said chill water for a period of time that is at least sufficient for the carcass to reach a pre-selected low temperature;
E) causing the chilled carcass to be removed from the chill tank; and
F) before packaging the chilled carcass, causing (i) the chilled carcass and (ii) a microbiocidal composition selected from the group consisting of a), b), c), and d) as described in claim 1 to come into contact with each other to effect microbiocidal control.

24. The improvement as in claim 23 wherein to cause the contacting in F), said microbiocidal composition is sprayed on said chilled carcass, and wherein after the contacting in F) the chilled carcass is rinsed at least once with clear water.

25. The improvement as in claim 23 wherein to cause the contacting in F), said chilled carcass is immersed in said microbiocidal composition, and wherein after the contacting in F) the chilled carcass is rinsed at least once with clear water.

26. The improvement as in claim 23 wherein the washing in C) is effected by use of an inside-outside washing apparatus through which the carcass is conveyed.

27. The improvement as in claim 26 wherein in said inside-outside washing apparatus, the interior cavity of said carcass is penetrated by a spray probe so that (i) contaminants together with (ii) microbiocidal composition that is sprayed into the interior cavity of the poultry by the probe, drain from the carcass.

28. The improvement as in claim 27 wherein in said inside-outside washing, pressurized sprays of the microbiocidal composition are applied to the exterior of the carcass so that the exterior of the carcass is thoroughly cleansed, and optionally the exterior of the carcass is also automatically mechanically scrubbed.

29. The improvement as in any of claims 1-7 wherein said bromine residual is in the range of about 3 to about 200 ppm (wt/wt) as total bromine.

30. The improvement as in any of claims 14-22 wherein the microbiocidal composition used in A), the microbiocidal composition used in C), and the microbiocidal composition used in D) each has, independently, a bromine residual in the range of about 3 to about 200 ppm (wt/wt) as total bromine.

31. In the processing of poultry for consumption as a meat product, the improvement which comprises:
A) causing an eviscerated poultry carcass to be subjected to inside-outside washing with a microbiocidal composition consisting essentially of
1) water having a microbiocidally-effective bromine residual derived from one or more N,N′-bromochloro-5,5-dialkylhydantoins; or
2) water having a bromine residual derived from at least one alkali or alkaline earth metal bromide and at least one alkali metal hypohalite or alkaline earth metal hypohalite; or
3) water having a bromine residual derived from a halogen stabilizer, a bromine source and an alkali metal base or alkaline earth metal base; or
4) water having a bromine residual derived from a mixture or combination of any two of 1), 2), and 3), or of all three of 1), 2) and 3); and
B) causing the carcass that was washed in A) to be placed in a chill tank and brought into contact with chill water which is composed of a microbiocidal composition selected from the group consisting of (1), (2), (3), and (4) as described in A) hereof, said carcass being in said chill water for a period of time that is at least sufficient for the carcass to reach a pre-selected low temperature,
the halogen residuals in A) and in B) being sufficient to provide microbiocidal activity without significant adverse effect upon the taste, odor, or appearance of the carcass.

32. The improvement as in claim 31 wherein a mechanically transported series of poultry carcasses is automatically transported into apparatus in which the poultry carcass is subjected to said inside-outside washing in A).

33. The improvement as in claim 32 wherein in said inside-outside washing, the interior cavity of a transported poultry carcass is penetrated by a spray probe so that (i) contaminants together with (ii) microbiocidal water solution that is sprayed into the interior cavity of the poultry by the probe, drain from the carcass.

34. The improvement as in claim 33 wherein in said inside-outside washing, pressurized sprays of the microbiocidal water solution are applied to the exterior of the carcass so that the exterior of the carcass is thoroughly cleansed, and optionally the exterior of the carcass is also automatically mechanically scrubbed.

35. The improvement as in any of claims 31-34 wherein the microbiocidal composition used in A) and in B) is a composition of 1).

36. The improvement as in any of claims 31-34 wherein the microbiocidal composition used in A) and in B) is a composition of 2).

37. The improvement as in any of claims 31-34 wherein the microbiocidal composition used in A) and in B) is a composition of 3).
